(19) United States
(12) Patent Application Publication
(10) Pub. No.: US 2007/0196295 A1
(43) Pub. Date: Aug. 23, 2007

(54) Cosmetic compositions and methods of making and using the compositions

(76) Inventors: Maggie Yun Cantwell, Cincinnati, OH (US); Howard A. Epstein, Cincinnati, OH (US)

Correspondence Address: MERCHANT & GOULD PC, P.O. BOX 2903, MINNEAPOLIS, MN, 55402-0903, US

(21) Appl. No.: 11/706,252

(22) Filed: Feb. 15, 2007

Related U.S. Application Data
(63) Continuation-in-part of application No. 11/355,152, filed on Feb. 16, 2006.

(51) Int. Cl. A61K 8/89

(52) U.S. Cl. 424/59

(57) ABSTRACT

Cosmetic compositions which, when applied to the skin, mask skin imperfections and provide more uniform skin tone are described. The compositions can be formulated to address the needs of users having a specific skin tone. The compositions include one or more pigments (e.g., iron oxides) and may also include reflective particles such as mica coated with iron oxide and/or titanium dioxide. The compositions can be oil-in-water emulsions comprising a discontinuous oil phase dispersed in a continuous aqueous phase. The compositions can also include emollients, one or more skin sheen additives, a film former (e.g., polycyclopentadiene) and isohexadecane. Compositions formulated for lighter skin tones can include one or more sunscreen agents. The compositions can be formulated to have high color transfer resistance. Methods of making the compositions and methods of treatment comprising applying the compositions to the skin are also described.

This application is a continuation-in-part of U.S. patent application Ser. No. 11/355,152, filed on Feb. 16, 2006, pending, the entirety of which is incorporated by reference herein.

### BACKGROUND OF THE INVENTION

1. Technical Field

This application relates generally to cosmetic compositions and to methods of making and using the cosmetic compositions. In particular, this application relates to moisturizing compositions comprising one or more pigments which, when applied to the skin, mask skin imperfections and provide more uniform skin tone and to methods of making and using these compositions.

2. Background of the Technology

Consumers are increasingly interested in improving the appearance of their skin. In particular, consumers want the skin on their body to look more even and more translucent and to look clear and unblemished. Consumers also want their skin to look and feel soft, smooth and unwrinkled. This desire for improved skin appearance is not limited to the skin of the face but also includes the neck and chest, the hands, the legs, the arms and anywhere else skin may be visible.

Consumers are currently using a variety of products and procedures to “fix” and/or prevent body skin problems. For example, scars and pregnancy rashes have been treated using glycolic serums and scar faders. Consumers with skin that is not sufficiently shiny, smooth or translucent or that need help with wrinkles, sun spots, pregnancy marks, etc. have employed resurfacing treatments (e.g., with alpha or beta hydroxys or peptides), microdermabrasion, exfoliating treatments, and skin fading treatments. Consumers with bumpy, dry or rough skin or with fine lines and wrinkles have employed exfoliants (e.g., sugar or salt). Consumers with uneven skin tone have employed fading creams. Consumers with lines and wrinkles have employed botox, collagen or laser resurfacing treatments. In addition, uneven skin tone has been treated using fading creams and visible veins have been treated using laser treatment. Tanning has been accomplished using self tanners and spray tanners. The procedures being employed by consumers also include plastic surgery and various dermatological approaches.

Many of these treatments have known shortcomings. For example, glycolics can cause irritation, especially for users having sensitive skin. Exfoliation treatments such as sugar and salt scrubs can be harsh to the skin. Dermatological treatments and collagen and botox injections can be costly. In addition, today's consumers want “immediate gratification” (i.e., they want to see and/or feel a difference in their skin immediately). In addition, today's consumers want products that will help conceal lines, wrinkles and other problems in addition to cleansing and moisturizing the skin.

Cosmetic compositions for application to the skin are described in the following publications. U.S. Pat. No. 5,853,712 discloses foundation compositions made by incorporating a mixture of pigments into a liquid base. U.S. Pat. No. 5,961,961 discloses cosmetic compositions containing an inorganic sunscreen agent such as titanium dioxide and an organic sunscreen agent. U.S. Pat. No. 5,997,887 discloses skin care compositions which comprise a charged particulate material dispersed in a hydrophilic carrier. U.S. Pat. No. 6,039,935 discloses a cosmetic sunscreen composition including at least one organic sunscreen, a cross-linked non-emulsifying siloxane elastomer, a volatile siloxane, and water. U.S. Pat. No. 6,284,228 B1 discloses a color blending system for foundation makeup compositions. U.S. Patent Publication No. 2004/0126337 Al discloses a composition comprising a lipophilic sunscreen, a copolymer of sodium acryloyldimethyltaurate and one or more acryls and an oil absorbent. U.S. Patent Publication No. 2005/0244348 A1 discloses a cosmetic or dermatological preparation for the cosmetic treatment of reddened skin comprising one or more green pigments, one or more white pigments and one or more UV protective filters. U.S. Patent Publication No. 2005/0031556 A1 discloses a light-protective cosmetic or dermatological preparation comprising at least one hydroxybenzophenone and at least one benzoxazole derivative. International Publication No. WO 96/03964 discloses foundation compositions made by incorporating a mixture of pigments into a liquid base. International Publication No. WO 97/03648 discloses composition for the topical delivery of active ingredients useful for treating conditions such as acne.

Although numerous compositions have been formulated for treating the skin, there still exists a need for improved cosmetic compositions which can subtly mask skin imperfections and impart a more uniform tone to the skin while also delivering emollients to the skin.

### SUMMARY OF THE INVENTION

According to a first embodiment, a cosmetic composition for application to the skin is provided which comprises: one or more pigments coated with a composition comprising an acrylic polymer; an emulsifier; optionally, one or more emollients; and optionally, one or more organic sunscreen agents; wherein each of the one or more pigments is present in the composition at a concentration such that the composition, when applied to the skin of a user, imparts a pigment to the skin which masks skin imperfections and imparts a more uniform tone to the skin. A method comprising applying a cosmetic composition as set above to the skin is also provided.

According to a second embodiment, a cosmetic composition for application to the skin is provided which comprises: one or more pigments; an emulsifier; optionally, one or more emollients; a film forming agent; and optionally, one or more organic sunscreen agents; wherein each of the one or more pigments are present in the composition at a concentration such that the composition, when applied to the skin of a user, imparts a pigment to the skin which masks skin imperfections and imparts a more uniform tone to the skin. A method comprising applying a cosmetic composition as set above to the skin is also provided.

According to a third embodiment, a cosmetic composition for application to the skin is provided which comprises: one or more pigments other than mica in a concentration of less than 0.09 percent by weight based on the total weight of the composition; an emulsifier; optionally, one or more emollients; and, optionally, one or more organic sunscreen agents; wherein each of the one or more pigments are present in the composition at a concentration such that the composition, when applied to the skin of a user, imparts a pigment to the skin which masks skin imperfections and imparts a more uniform tone to the skin. A method comprising applying a cosmetic composition as set above to the skin is also provided.

According to a fourth embodiment, a method of making an oil-in-water emulsion is provided which comprises: mixing an aqueous phase with an oil phase comprising an oil-in-water emulsifier and, optionally, one or more emollients at a temperature greater than 70° C.; cooling the resulting mixture; mixing one or more iron oxide pigments into the mixture at a temperature at or below 60° C. (e.g., 53° C.-57° C.); and mixing hydrogenated polycyclopentadiene and isohexadecane into the resulting mixture at a temperature at or below 55° C. (e.g., 50° C.-55° C.). The method as set forth above can further comprise mixing one or more reflective pigments into the mixture subsequent to mixing hydrogenated polycyclopentadiene and isohexadecane into the resulting mixture.

### DETAILED DESCRIPTION OF THE INVENTION

Skin care issues can typically be grouped according to the skin tone of the user. Thus, consumers having a particular skin tone will tend to have similar concerns or issues with the appearance of their skin. Skin tones can be classified using the Fitzpatrick Phototype Scale {J. Amer. Acad. of Dermatology: 46(2) Supplement (2002); 48(6) S139-S142 (S2003)}. The Fitzpatrick Phototype Scale divides skin types into six categories ranging from ivory white to dark brown or black. The following table illustrates some general characteristics of each of the Fitzpatrick skin phototypes.

| | I | II | III | IV | V | VI |
|---|---|---|---|---|---|---|
| Unexposed | Ivory White | White | White | Beige or | Moderate | Dark brown |
| Skin Color | | | | lightly | brown or | or black |
| | | | | tanned | tanned | |
| Sensitivity | Very | Very | Quite | Moderately | Minimally | Least |
| to UV | Sensitive | Sensitive | Reactive | Reactive | Sensitive (+) | Sensitive |
| (Sunlight) | (++++) | (+++/++++) | (+++) | (++) | | (0-±) |
| Sunburn | Burns easily/ | Burns easily/ | Burns and | Burns | Rarely burns/ | Never burns/ |
| History | Never tans | tans with | tans | minimally/ | tans | tans |
| | | difficulty | moderately | Tans easily | profusely | profusely |
| Photoaging | Strong/Early | Strong/ | Moderate to | Moderate to | Slow, gradual, | Slow, gradual |
| | onset | Early onset | strong | low/not | and low | and minimum |
| | | | | excessive | | |
| Ethnicity | Northern | European/ | Central/ | Asian/ | African | African |
| Examples | European/ | Native | Eastern/ | Filipino | | |
| | Celtic | American | Southern | | | |
| | | | Europe | | | |

The compositions described herein can be skin care lotions that can be applied to the skin in a conventional manner. The formulations described herein can be customized to address the specific skin care needs of diverse skin types. The formulations can be “customized” to immediately improve the appearance and texture of skin while other ingredients in the formulations continue to provide additional benefits over time with continued use of the product. The formulations described herein can therefore be considered hybrid products that incorporate the best properties of a makeup, a skin care emollient lotion and a fortifying agent (e.g., a sunscreen).

According to one embodiment, the compositions described herein are oil-in-water emulsions. These oil-in-water emulsions, however, can contain a non-ionic water-in-oil emulsifier based on silicone (Cetyl PEG/PPG-10/1 Dimethicone). The incorporation of Cetyl PEG/PEG-10/1 Dimethicone into the oil phase was found to improve resistance to color transfer from skin to clothing and provide improvement to the “feel” of the formulation when applied to skin. Further, when a sunscreen was added to formulations including Cetyl PEG/PEG-10/1 Dimethicone, it was found that the level of sunscreen required to achieve an SPF of 16 could be dramatically reduced. This effect was achieved with relatively low levels of the Cetyl PEG/PEG-10/1 Dimethicone (i.e., 0.4 wt. % based on the total weight of the composition). In contrast, the manufacturer recommends use levels of 1.5-2.5 wt. % of Cetyl PEG/PEG-10/1 Dimethicone.

The compositions can also be formulated as water-in-oil emulsions.

The compositions described herein can also include pigments (e.g., iron oxide pigments). The pigments may be surface treated with an acrylic polymer. For example, the pigments can be surface treated with a composition comprising water, an acrylate/octyl acrylate copolymer, a sodium acrylate/sodium acryloydimethyl taurate copolymer and aminomethyl propanol. A material of this type is available from Kobo Products, Inc. It was found that iron oxides with other surface treatments (e.g., Sympholight which is an iron oxide/silica) did not result in compositions having comparable color transfer resistance. Although pigments having acrylic polymer surface treatments are preferred, pigments having no surface treatment or pigments having other surface treatments can also be used.

The iron oxide pigments included in the formulations can help even natural skin tone variation found within each general skin type. Unexpectedly, it was found that the iron oxide pigments, when used in the base formulation, can have a strong color impact on the mass tone of the skin with minimal color transfer from skin to clothing.

In addition to the above, color transfer from the body to clothing can be further minimized by incorporation of hydrogenated polycyclopentadiene (e.g., Koboguard 5400 IDD). This material is a film-forming low molecular weight polymer that is extremely stick and tacky. For this reason it is not commonly used in body lotions. To remove the sticky, tacky property the formulation and further reduce color transfer, isohexadecane (e.g., Permethyl 101A) can be added to the formulations. The hydrogenated polycyclopentadiene and isohexadecane can be pre-mixed and added to the formulation after the oil and water phases are combined and the resulting emulsion has formed. The hydrogenated polycyclopentadiene and isohexadecane can be added to the emulsion when the emulsion is at a temperature below 50° C. In this manner, a further reduction in pigment transfer can be realized.

Other film forming agents can also be used. By film forming agents, it is meant materials which, upon drying, produce a continuous film on skin, hair, or nails. Exemplary film forming agents which can be used in the compositions are set forth in the following table.

Acrylamide/Ammonium Acrylate Copolymer
Acrylamides Copolymer
Acrylamides/DMAPA Acrylates/Methoxy PEG
Methacrylate Copolymer
Acrylamide/Sodium Acrylate Copolymer
Acrylamidopropyltrimonium Chloride/Acrylamide
Copolymer
Acrylamidopropyltrimonium Chloride/Acrylates
Copolymer
Acrylatesl Acetoacetoxyethyl Methacrylate
Copolymer
Acrylates/Acrylamide Copolymer
Acrylates/Ammonium Methacrylate Copolymer
Acrylates Copolymer
Acrylates/Diacetoneacrylamide Copolymer
Acrylates/Dimethicone Copolymer
Acrylates/Dimethylaminoethyl Methacrylate
Copolymer
Acrylates/Hydroxyesters Acrylates Copolymer
Acrylates/Octylacrylamide Copolymer
Acrylates/Octyl Acrylate Copolymer
Acrylates/PVP Copolymer
Acrylates/VA Copolymer
Acrylates/VA Crosspolymer
Acrylic Acid/Acrylonitrogens Copolymer
Adipic Acid/CHDM/MA/Neopentyl Glycol/
Trimellitic Anhydride Copolymer
Adipic Acid/Diethylene Glycol/Glycerin
Crosspolymer
Adipic Acid/Diethylenetriamine Copolymer
Adipic Acid/Dimethylaminohydroxypropyl
Diethylenetriamine Copolymer
Adipic Acid/Epoxypropyl Diethylenetriamine
Copolymer
Adipic Acid/Fumaric Acid/Phthalic
Acid/Tricyclodecane Dimethanol Copolymer
Adipic Acid/Isophthalic Acid/Neopentyl Glycol!
Trimethylolpropane Copolymer
Adipic Acid/Neopentyl GlycollTrimeliitic
Anhydride Copolymer
Albumen
Allyl Stearate/VA Copolymer
Aminoethylacrylate Phosphate/Acrylates
Copolymer
Ammonium Acrylates/Acrylonitrogens Copolymer
Ammonium Acrylates Copolymer
Ammonium Alginate
Ammonium Polyacrylate
Ammonium Styrene/Acrylates Copolymer
Ammonium VA/Acrylates Copolymer
AMP-Acrylates Copolymer
AMP-Acrylates/Diacetoneacrylamide Copolymer
AMP-Acrylates/Dimethylaminoethylmethacrylate
Copolymer
AMPD-Acrylates/Diacetoneacrylamide Copolymer
Balsam Canada (Abies Balsamea)
Balsam Copaiba (Copaifera Officinalis)
Balsam Oregon (Pseudotsuga Menziesi)
Balsam Peru (Myroxylon Pereirae)
Balsam Tolu (Myroxylon Balsamum)
Benzoic Acid/Phthalic Anhydride/Pentaerythritol/
Neopentyl Glycol/Palmitic Acid Copolymer
Benzoin (Styrax Benzoin) Gum
Butadiene/Acrylonitrile Copolymer
Butoxy Chitosan
Butyl Acrylate/Hydroxyethyl Methacrylate
Copolymer
Butyl Acrylate/Styrene Copolymer
Butylated Polyoxymethylene Urea
Butylated PVP
Butyl Benzoic Acid/Phthalic
Anhydride/Trimethylolethane Copolymer
Butyl Ester of Ethylene/MA Copolymer
Butyl Ester of PVM/MA Copolymer
Calcium Carboxymethyl Cellulose
Calcium Carrageenan
Calcium/Sodium PVM/MA Copolymer
C1-5 Alkyl Galactomannan
Carboxybutyl Chitosan
Carboxymethyl Chitosan
Carboxymethyl Chitosan Succinamide
Carboxymethyl Dextran
Carboxymethyl Hydroxyethylcellulose
Castor Oil/IPDI Copolymer
Cellulose Acetate
Cellulose Acetate Butyrate
Cellulose Acetate Propionate
Cellulose Acetate Propionate
Carboxylate Cellulose Gum
Cetearyl Dimethicone/Vinyl Dimethicone
Crosspolymer
Chitosan
Chitosan Adipate
Chitosan Ascorbate
Chitosan Formate
Chitosan Glycolate
Chitosan Lactate
Chitosan PCA
Chitosan Salicylate
Chitosan Succinamide
Collodion
Copal
Corn Starch/Acrylamide/Sodium Acrylate
Copolymer
DEA-Styrene/Acrylates/DVB Copolymer
Diethylene Glycolamine/Epichlorohydrin/
Piperazine Copolymer
Diglycol/CHDM/Isophthalates/SIP Copolymer
DiglycollIsophthatates/SIP Copolymer
Dihydroxyethyl Tallowamine/IPDI Copolymer
Dilinoleyl Alcohol/IPDI Copolymer
Dimethicone/Sodium PG-Propyldimethicone
Thiosulfate Copolymer
Dimethiconol/IPDI Copolymer
DMAPA Acrylates/Acrylic Acid/Acrylonitrogens
Copolymer
Dodecanedioic Acid/Cetearyl Alcohol/Glycol
Copolymer
Ethylcellulose
Ethylene/Acrylic Acid Copolymer
Ethylene/Acrylic Acid/VA Copolymer
Ethylene/Calcium Acrylate Copolymer
Ethylene/MA Copolymer
Ethylene/Magnesium Acrylate Copolymer
Ethylene/Methacrylate Copolymer
Ethylene/Propylene Copolymer
Ethylene/Sodium Acrylate Copolymer
Ethylene/VA Copolymer
Ethylene/Zinc Acrylate Copolymer
Ethyl Ester of PVM/MA Copolymer
Flexible Collodion
Galactoarabinan
Glycereth-7 Hydroxystearate/IPDI Copolymer
Glyceryl Polyacrylate
Glyceryl Polymethacrylate
Gutta Percha
Hydrogenated Rosin
Hydrogenated Styrene/Butadiene Copolymer
Hydrolyzed Wheat Protein/Dimethicone
Copolyol Phosphate Copolymer
Hydroxybutyl Methylcellulose
Hydroxyethylcellulose
Hydroxyethyl Chitosan
Hydroxyethyl Ethylcellulose
Hydroxypropylcellulose
Hydroxypropyl Chitosan
Hydroxypropyl Guar
Hydroxypropyl Methylcellulose
Isobutylene/MA Copolymer
Isobutylene/Sodium Maleate Copolymer
Isopropyl Ester of PVM/MA Copolymer
Lauryl Acrylate/VA Copolymer
Lauryl Methacrylate/Glycol Dimethacrylate
Copolymer
Maltodextrin
Mannan
Methacryloyl Ethyl Betaine/Acrylates
Copolymer
Methyl Methacrylate Crosspolymer
Nitrocellulose
Nylon-12/6/66 Copolymer
Octadecene/MA Copolymer
Octylacrylamide/Acrylates/Butylaminoethyl
Methacrylate Copolymer
Phthalic Anhydride/Adipic Acid/Castor Oil/
Neopentyl Glycol/PEG-
3/Trimethylolpropane Copolymer
Phthalic Anhydride/Benzoic Acid/
Trimethylolpropane Copolymer
Phthalic Anhydride/Butyl Benzoic
Acid/Propylene Glycol Copolymer
Phthalic Anhydride/Glycerin/Glycidyl
Decanoate Copolymer
Phthalic Anhydride/Trimellitic
Anhydride/Glycols Copolymer
Piperylene/Butene/Pentene Copolymer
Polyacrylamide
Polyacrylamidomethylpropane Sulfonic Acid
Polyacrylic Acid
Polybeta-Alanine
Polybeta-alanine/Glutaric Acid Crosspolymer
Polybutyl Acrylate
Polybutylene Terephthalate
Polychlorotrifluoroethylene
Polydiethyleneglycol Adipate/IPDI
Copolymer
Polydimethylaminoethyl Methacrylate
Polyethylacrylate
Polyethylene
Polyethylene Terephthalate
Polyethylglutamate
Polyethylmethacrylate
Polyglucuronic Acid
Polyisobutene
Polylysine
Polymethacrylamidopropyltrimonium
Methosulfate
Polymethacrylic Acid
Polymethyl Acrylate
Polymethylglutamate
Polymethyl Methacrylate
Polyoxyisobutylene/Methylene Urea Copolymer
Polyoxymethylene Melamine
Polypentene
Polyperfluoroperhydrophenanthrene
Poly-p-Phenylene Terephthalamide
Polyquaternium-1
Polyquaternium-2
Polyquaternium-4
Polyquaternium-5
Polyquaternium-6
Polyquaternium-7
Polyquaternium-8
Polyquaternium-9
Polyquaternium-10
Polyquaternium-11
Polyquaternium-12
Polyquaternium-13
Polyquaternium-14
Polyquaternium-15
Polyquaternium-16
Polyquaternium-17
Polyquaternium-18
Polyquaternium-19
Polyquaternium-20
Polyquaternium-22
Polyquaternium-24
Polyquaternium-27
Polyquaternium-28
Polyquaternium-29
Polyquaternium-30
Polyquaternium-31
Polyquaternium-32
Polyquaternium-33
Polyquaternium-34
Polyquaternium-35
Polyquaternium-36
Polyquaternium-37
Polyquaternium-39
Polyquaternium-43
Polyquaternium-44
Polyquaternium-45
Polyquaternium-46
Polyquaternium-47
Polysilicone-6
Polysilicone-8
Polysilicone-11
Polystyrene
Polyvinyl Acetate
Polyvinyl Alcohol
Polyvinyl Butyral
Polyvinylcaprolactam
Polyvinyl Chloride
Polyvinyl Imidazolinium Acetate
Polyvinyl Laurate
Polyvinyl Methyl Ether
Polyvinyl Stearyl Ether
Potassium Carbomer
Potassium Carrageenan
PPG-12/SMDI Copolymer
PPG-7/Succinic Acid Copolymer
PPG-26/TDI Copolymer
PVM/MA Copolymer
PVM/MA Decadiene Crosspolymer
PVP
PVP/Dimethiconylacrylate/Polycarbamyl/
Polyglycol Ester
PVP/Dimethylaminoethylmethacrylate Copolymer
PVP/Dimethylaminoethylmethacrylate/
Polycarbamyl Polyglycol Ester
PVP/Eicosene Copolymer
PVP/Hexadecene Copolymer
PVP/Polycarbamyl Polyglycol Ester
PVP/VA Copolymer
PVP/VA/Itaconic Acid Copolymer
PVP/VA/Vinyl Propionate Copolymer
Quaternium-22
Rosin
Rubber Latex
Serum Albumin
Shellac
Sodium Acrylates/Acrolein Copolymer
Sodium Acrylates Copolymer
Sodium Acrylate/Vinyl Alcohol Copolymer
Sodium Carbomer
Sodium Carboxymethyl Chitin
Sodium Carboxymethyl Starch
Sodium Carrageenan
Sodium C4-12 Olefin/Maleic Acid Copolymer
Sodium DVB/Acrylates Copolymer
Sodium Isooctylene/MA Copolymer
Sodium MA/Diisobutylene Copolymer
Sodium Polyacrylate
Sodium Polymethacrylate
Sodium Polystyrene Sulfonate
Sodium PVM/MA/Decadiene Crosspolymer
Sodium Styrene/Acrylates Copolymer
Sodium Tauride Acrylates/Acrylic Acid/
Acrylonitrogens Copolymer
Starch/Acrylates/Acrylamide Copolymer
Starch Diethylaminoethyl Ether
Stearamidopropyl Dimethicone
Steareth-10 Allyl Ether/Acrylates Copolymer
Stearylvinyl Ether/MA Copolymer
Styrene/Acrylates/Acrylonitrile Copolymer
Styrene/Acrylates/Ammonium Methacrylate
Copolymer
Styrene/Acrylates Copolymer
Styrene/Allyl Benzoate Copolymer
Styrene/DVB Copolymer
Styrene/Isoprene Copolymer
Styrene/MA Copolymer
Styrene/Methylstyrene/Indene Copolymer
Styrene/PVP Copolymer
Styrene/VA Copolymer
Sucrose Benzoate/Sucrose Acetate Isobutyrate/
Butyl Benzyl Phthalate Copolymer
Sucrose Benzoate/Sucrose Acetate
Isobutyrate/Butyl Benzyl Phthalate/Methyl
Methacrylate Copolymer
Sucrose Benzoate/Sucrose Acetate Isobutyrate
Copolymer
TEA-Acrylates/Acrylonitrogens Copolymer
Terephthatic Acid/Isophthalic Acid/Sodium
Isophthalic Acid Sulfonate/Glycol Copolymer
Tosylamide/Epoxy Resin
Tosylamide/Formaldehyde Resin
Tragacanth (Astragalus Gummifer) Gum
Tricontanyl PVP
Trimethylpentanediol/Isophthalic Acid/Trimellitic
Anhydride Copolymer
Tromethamine Acrylates/Acrylonitrogens
Copolymer
VA/Butyl Maleate/Isobornyl Acrylate Copolymer
VA/Crotonates Copolymer
VA/Crotonates/Methacryloxybenzophenone-1
Copolymer
VA/Crotonates/Vinyl Neodecanoate Copolymer
VA/Crotonates/Vinyl Propionate Copolymer
VA/Crotonic Acid/PEG-20M Copolymer
VA/DBM Copolymer
VA/Isobutyl Maleate/Vinyl Neodecanoate
Copolymer
VA/Vinyl Butyl Benzoate/Crotonates Copolymer
Vinyl Acetate
Vinyl Caprolactam/PVP/Dimethylaminoethyl
Methacrylate Copolymer
Yeast Betaglucan
Yeast Polysaccharides
Zein

The above listing of film forming agents is not exhaustive and other film forming agents can also be used. Further, combinations of film forming agents can also be used.

When one or more sunscreen agents (e.g., oxybenzone and/or octylmethoxycinnamate) were added to a formulation comprising hydrogenated polycyclopentadiene and isohexadecane, it was discovered that an SPF of 16 could be achieved with relatively low levels of the sunscreen agents. While not wishing to be bound by theory, it is believed that this phenomenon may be related to the solubility properties of isohexadecane. Moreover, oxybenzone is insoluble in isohexadecane and octylmethoxycinnamate is soluble in isohexadecane. It is believed that the relative solubilites of these ingredients has sufficiently shifted the action spectra of the sunscreens resulting in more efficient SPF results. This is desirable in a lotion containing sunscreens with respect to both toxicology and improved aesthetic performance.

Reflective pigments can also be incorporated into the compositions. These pigments can be titanium/mica and or iron oxide/mica blends with no additional surface coating. The blend of reflective pigments employed can vary depending on the skin type of the user. The particle size of the reflective pigments can range from 4 to 75 microns.

The compositions can be oil-in-water emulsions comprising a discontinuous oil phase dispersed in a continuous aqueous phase. The compositions can also be in the form of lotions.

As set forth above, the compositions include various color pigments the concentrations of which can be adjusted for the skin tone of the user. For example, the compositions can include a combination of red, black and yellow pigments. Exemplary pigments include iron oxides. The pigments (e.g., iron oxides) can be surface treated. According to one embodiment, the compositions comprise iron oxide pigments surface treated with a composition comprising an acrylic polymer. Compositions formulated for the lighter skin tones (e.g., Fitzpatrick I and II phototypes) can be formulated with a pigment package which minimizes the appearance of red splotches on the skin. Compositions formulated for intermediate skin tones (e.g., Fitzpatrick Phototype IV) can be formulated with a pigment package which highlights the natural skin color and hue of the user. Compositions formulated for the darker skin tones (e.g., Fitzpatrick Phototypes V and VI) can be formulated with a pigment package which provides enhanced sheen and luster to the skin of the user.

Exemplary pigments include iron oxides such as hydrated ferric oxide (FeHO₂), ferric oxide (Fe₂O₃), ferrosoferric oxide (Fe₃O₄). As set forth above, the iron oxide pigments can be coated with a composition comprising an acrylic polymer. An exemplary acrylic polymer is an ethyl methacrylate/n-butyl acrylate/2-methylhexyl acrylate copolymer. A suitable material of this type is available from Kobo Products, Inc. The use of iron oxide pigments coated with an acrylic polymer increases the transfer resistance of the compositions.

As also set forth above, the compositions can also include reflective pigments such as mica. The mica can be coated (e.g., with titanium dioxide or iron oxide) such that the mica pigments act as light diffusers. The compositions formulated for the lighter skin tones (e.g., Fitzpatrick Phototypes I and II) can include red and silver mica pigments whereas the compositions formulated for the darker skin tones (e.g., Fitzpatrick Phototypes V and VI) can include copper, bronze and gold mica pigments. Compositions formulated for intermediate skin tones (e.g., Fitzpatrick Phototype IV) can include amber and gold mica pigments.

Color transfer resistancy (e.g., from the skin of the user to clothing) can be further enhanced by including various polymers (e.g., film formers) in the compositions. Exemplary film forming polymers include hydrogenated polycyclopentadiene.

Exemplary compositions for the lighter skin tones of the Fitzpatrick Skin Phototype Scale (e.g., Types I-V) can also include one or more sunscreen agents. Exemplary sunscreen agents include but are not limited to octyl methoxycinnamate, octylsalicylate, oxybenzone and meradimate. Compositions formulated for the lighter skin tones (e.g., Fitzpatrick I and II phototypes) can also be formulated with antioxidants to reverse the effects of ultraviolet radiation as well as other insults to the skin.

Skin care compositions can also include a humectant (e.g., butylene glycol), a thickener (e.g., ammonium acryloyidimethyltaurate/vinyl pyrrolidone copolymer or hydroxyethyl acrylate/sodium acryloyldimethyl taurate copolymer), a chelating agent (e.g., disodium EDTA), a preservative (e.g., methylparaben, propylebaraben, DMDM hydantoin or combinations thereof), a non-ionic emulsifier (e.g., ceteareth-20), and/or a fatty alcohol (e.g., cetearyl alcohol, cetyl alcohol or combinations thereof).

Exemplary compositions for the darker skin tones (e.g., Fitzpatrick Skin Phototypes V and VI) can also include dicaprylyl ether. An exemplary concentration of dicaprylyl ether is about 3.0 weight percent based on the total weight of the composition.

The compositions can also include a fragrance.

The compositions can also include a matifying agent. An exemplary matifying agent is a dimethicone/cyclopentasiloxane gel made by dispersing oil swellable polydimethylsiloxane particles in a mixture of dimethicone and cyclopentasiloxane. A material of this type is available from Grant Industries and is sold under the tradename Gransil.

The compositions can include an alkylated polyether polydimethylsiloxane compound. An exemplary alkylated polyether polydimethylsiloxane compound has the following general structure: 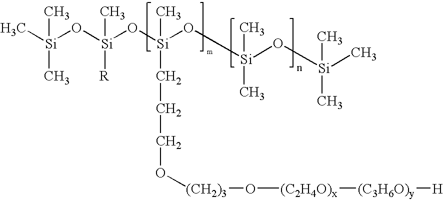
wherein R is an alkyl group (e.g., a cetyl group) and wherein m, n, x and y are integers. A material of this type is sold under the trademark Abil® EM 90 (INCI name Cetyl PEG/PPG-10/1 Dimethicone).

The compositions can also include an emollient package. Representative emollient packages for compositions for fair skin tones (e.g., Fitzpatrick Skin Phototypes I and II), medium skin tones (e.g., Fitzpatrick Skin Phototypes II and III), tan skin tones (e.g., Fitzpatrick Skin Phototypes III-V) and deep skin tones (e.g., Fitzpatrick Skin Phototypes V and VI) are set forth in the following table.

| | FAIR | MEDIUM | TAN | DEEP |
|---|---|---|---|---|
| Butylene Glycol | 3.0% | 3.0% | 3.0% | 3.0% |
| Bernel Ester DID | 0.5% | 0.5% | 0.5% | 0.5% |
| (Diisopropyl Dimer Dilinoleate) | | | | |
| Finsolv TN (C12-15 Alkyl | 3.0% | 3.0 | — | — |
| Benzopate) | | | | |
| Octyldodecyl Myristate | — | — | 1.5% | 1.5% |
| Ethylhexyl Myristate | — | — | 1.5% | 1.5% |
| Shea Butter (Butyrospermum parkii) | — | — | — | 5.0% |

The compositions may also contain additional materials such as, for example, fragrances, fillers, preservatives, antioxidants, and emulsifiers. Exemplary emulsifiers include any of the surfactants (e.g., nonionic, anionic, cationic or zwitterionic) used to form emulsions. Suitable emulsifiers also include polymeric emulsifiers such as Pemulen® emulsifiers available from Noveon. These materials are crosslinked copolymers of acrylic acid and C₁₀-C₃₀ alkyl acrylate.

Exemplary compositions A, B, C, D and E are set forth in the following table.

| | | A | B | C | D | E |
|---|---|---|---|---|---|---|
| INCI Name | CAS No. | % WAV | % WAV | % WAV | % WAV | % WAV |
| Water | 7732-18-5 | q.s. to | q.s. to | q.s. to | q.s. to | q.s. to |
| | | 100% | 100% | 100% | 100% | 100% |
| Butylene Glycol | 107-88-0 | 3.00 | 3.00 | 3.00 | 3.00 | 3.00 |
| Ammoniumn | Proprietary | 0.35 | 0.35 | 0.35 | 0.35 | 0.35 |
| Acryloydimethyltaurate/ | | | | | | |
| VP Copolymer | | | | | | |
| Disodium EDTA | 006381-92-6 | 0.05 | 0.05 | 0.05 | 0.05 | 0.05 |
| Methylparaben | 99-76-3 | 0.20 | 0.20 | 0.20 | 0.20 | 0.20 |
| Propylparaben | 94-13-3 | 0.1 | 0.10 | 0.10 | 0.10 | 0.10 |
| Cetearyl Alcohol | 8005-44-5; 67762-27-0; | 3.5 | 3.5 | 3.5 | 3.5 | 3.5 |
| | 67762-30-5 | | | | | |
| Ceteareth-20 | 68439-49-6 | 0.5 | 0.5 | 0.5 | 0.5 | 0.5 |
| Cetyl Alcohol | 36653-82-4 | 0.5 | 0.5 | 0.5 | 0.5 | 0.5 |
| Glyceryl Stearate & | 123-94-4; 11099-07-3; | 3.5 | 3.5 | 3.5 | 3.5 | 3.5 |
| PEG 100 Stearate | 31566-31-1; 85666-92; | | | | | |
| | 8; 9004-99-3 | | | | | |
| Dicaprylyl Ether | 629-82-3 | — | — | — | 3.0 | — |
| Cetyl PEG/PPG-10/1 | Proprietary | 0.4 | 0.4 | 0.4 | | 0.4 |
| Dimethicone | | | | | | |
| Diisopropyl Dimer | 103213-20-3 | 0.5 | 0.5 | 0.5 | | 0.5 |
| Dilinoleate | | | | | | |
| C12-15 Alkyl | 68411-27-8 | 3.0 | 3.00 | 3.00 | — | 3.00 |
| Benzoate | | | | | | |
| Polydimethylsiloxane | 63148-62-9 | 0.75 | 0.75 | 0.75 | 0.75 | 0.75 |
| Octinoxate (Octyl | 5466-77-3 | 7.5 | 7.5 | 6.5 | — | 6.5 |
| Methoxycinnamate) | | | | | | |
| Octisalate | 118-60-5 | 4.0 | 4.0 | — | — | — |
| (Octylsalicylate) | | | | | | |
| Oxybenzone | 131-57-7 | 3.0 | 3.0 | — | — | — |
| (Benzophenone 3) | | | | | | |
| Meradimate | 134-09-8 | — | — | 5.0 | — | 5.0 |
| Montmorillonite, | 1318-93-0; 7732-18-5; | — | — | — | — | 1.0 |
| Water, Picea Excelsa | 91770-69-3; 13463-67-7 | | | | | |
| Extract, Titanium Dioxide | | | | | | |
| DMDM Hydantoin | 6440-58-0 | 0.4 | 0.4 | 0.4 | 0.4 | 0.4 |
| Hydrogenated | 68132-00-3; 141-70-8 | 2.5 | 2.5 | 2.5 | 2.5 | 2.5 |
| Polycyclopentadiene | | | | | | |
| (and) Isododecane | | | | | | |
| Isohexadecane | 4390-04-9 | 4.5 | 4.5 | 4.5 | 4.5 | 4.5 |
| Dimethicone (and) | 9006-65-9; 541-02-6; | 0.3 | 0.3 | 0.3 | 0.3 | 0.3 |
| Cyclopentasiloxine | 63394-02-5 | | | | | |
| (and) Polysilicone-II | | | | | | |
| Water (and) Acrylates/ | 7732-18-5; 42398-14-1; | 0.05 | 0.05 | 0.05-0.6 | 0.05-0.6 | 0.05 |
| Ethylhexyl Acrylate | 20344-49-4; 77019-71-1; | | | | | |
| Copolymer (and) Iron | 20344-49-4; 124-68-5 | | | | | |
| Oxide (C.I. 77492) (and) | | | | | | |
| Sodium Acrylate/Sodium | | | | | | |
| Acryloyldimethyl Taurate | | | | | | |
| Copolymer (and) | | | | | | |
| Aminomethyl Propopanol | | | | | | |
| Water (and) Ethyl | 7732-18-5; 42398-14-1; | 0.02 | 0.02 | 0.02-0.6 | 0.02-0.6 | 0.02 |
| Methacrylate/N-Butyl | 1309-37-1; 7709-71-7; | | | | | |
| Acrylate/2-Methylhexyl | 124-68-5 | | | | | |
| Acrylate Copolymer (and | | | | | | |
| Ferric Oxide (and) Sodium | | | | | | |
| Acrylodimethyl Taurate | | | | | | |
| Copolymer (and) 2-Amino-2- | | | | | | |
| Methyl-1-Propanol | | | | | | |
| Water (and) Acrylates/ | 7732-18-5; 42398-14-1; | 0.01 | 0.01 | 0.01-0.6 | 0.01-0.6 | 0.01 |
| Ethylhexyl Acrylate | 1317-61-9; 77019-71-7 | | | | | |
| Copolymer (and) Iron | | | | | | |
| Oxide (C.I. 77499) (and) | | | | | | |
| Sodium Acrylate/Sodium | | | | | | |
| Acryloyldimethyl Taurate | | | | | | |
| Copolymer (and) | | | | | | |
| Aminomethyl Propopanol | | | | | | |
| Mica (and) titanium | 12001-26-2; 13463-67-7 | 0.1 | 0.1 | | | 0.1 |
| dioxide | | | | | | |
| Titanium dioxide | 12001-26-2; 13463-67-7 | 0.05 | 0.05 | | | 0.05 |
| (and) mica | | | | | | |
| Stryene/Acrylates | Proprietery; 1336-21-6; | — | 1.0-3.0 | — | — | — |
| Copolymer | 7732-18-5 | | | | | |
| Titanium Dioxide, | 12001-26-2; 13463-67- | 0-0.6 | 0-0.6 | 0-0.6 | 0-0.6 | 0-0.6 |
| Mica, Iron Oxide(s) | 713097-37-1; 1309-38-2; | | | | | |
| | 1317-61-9; 1332-37-2; | | | | | |
| | 1345-25-1; 12227-89-3; | | | | | |
| | 20344-49-4; 51274-00-1; | | | | | |
| | 52357-70-7; 64294-91-3 | | | | | |

As can be seen from the above table, each of the compositions includes a plurality of pigments (i.e., iron oxides treated with an acrylic polymer), a water-in-oil emulsifier, and an emollient package.
Compounding Procedure

An exemplary compounding procedure is described below.

The aqueous phase is heated to a temperature over 70° C. (e.g., 75-78° C.). The water phase can include water, butylene glycol, Disodium EDTA, Aristoflex AVC and propyl paraben and methylparaben.

The oil phase is heated to about 80° C. The oil phase can include glyceryl stearate & PEG 100 stearate, cetearyl alcohol, ceteareth-20, cetyl alcohol, Cetyl PEG/PPG10-1 Dimethicone, Bernel ester DID, Shea butter, octyl myristate, ethylhexyl isononate, and C12-15 alkyl benzoate.

After the oil phase is added to the aqueous phase at the above temperatures and the resulting mixture is cooled to about 50-55° C., the iron oxide pigments are added. The iron oxide pigments can be pre-mixed in water. Pre-mixing in water can help to further hydrate the polymer to minimize polymer specs in the final batch.

The silicone/polymer/silicone elastomer/solvent phase is premixed at room temp. This phase can include Koboguard 5400 IDD, Permethyl 101A, Dimethicone 200 5 CS, and Gransil KO.

This phase is added at a temperature at or below 55° C. (e.g., 50-55° C.) after the emulsion has formed. In this manner, reduced pigment transfer as well as a unique feel of the formulation can be achieved.

After the above is well mixed, the mica effect colors can be added (e.g., at a temperature of 45-50° C.), followed by the DMDM Hydantoin (a preservative) and the fragrance (e.g., at a temperature of 400° C.).

Additional Exemplary Compositions

Additional exemplary compositions for fair skin tones (e.g., Fitzpatrick Phototypes I and II), medium skin tones (e.g., Fitzpatrick Phototypes II and III), tan skin tones (e.g., Fitzpatrick Phototypes III-V), and deep skin tones (e.g., Fitzpatrick Phototypes V and VI) are described below.

Exemplary Composition for Fair Skin Tones

An exemplary composition for fair skin tones is set forth in the following table.

| PART | DESCRIPTION | % wt. |
|---|---|---|
| A | Deionized Water | 57.970% |
| | Butylene Glycol | 3.000% |
| | Disodium EDTA | 0.050% |
| | Aristoflex AVC (ammonium | 0.350% |
| | acryloyldimethyltaurate/VP copolymer) | |
| | Propylparaben NF | 0.100% |
| | Methylparaben | 0.200% |
| B1 | Glyceryl Stearate & PEG 100 Stearate | 3.500% |
| | Cetearyl Alcohol | 3.500% |
| | Ceteareth-20 | 0.500% |
| | Cetyl Alcohol | 0.500% |
| | Abil EM-90 (Cetyl PEG/PPG-10/1 Dimethicone) | 0.400% |
| | Bernel Ester DID (Diisopropyl Dimer Dilinoleate) | 0.500% |
| B2 | Finsolv TN (C12-15 Alkyl Benzoate) | 3.000% |
| | Octyl Methoxycinnamate/Esc 557 | 3.750% |
| | Octyl Salicylate/Esc 587 | 2.000% |
| | Oxybenzone/Esc 567 | 1.500% |
| C | Deionized Water | 4.000% |
| | Iron Oxide (Kobo Yellow WSJ20EYAMP) | 0.050% |
| | Iron Oxide (Kobo Red WSJ22ERAMP) | 0.020% |
| | Iron Oxide (Kobo Black) | 0.010% |
| | Deionized Water (Rinse) | 1.000% |
| D | Koboguard 5400 IDD (Hydrogenated | 2.500% |
| | Polycyclopentadiene and Isododecane) | |
| | Permethyl 101A (isohexadecane) | 4.500% |
| | Dimethicone 200 5 CS | 0.750% |
| | Gransil KO (dimethicone and cyclopentasiloxane | 0.300% |
| | and polysilicone-11) | |
| E | Deionized Water | 4.000% |
| | KTZ Interference Red | 0.100% |
| | KTZ Fine White | 0.050% |
| | Deionized Water (Rinse) | 1.000% |
| F | DMDM Hydantoin | 0.400% |
| | Fragrance | 0.500% |
| | | 100.00% |

The above composition can be made by a procedure as set forth below.

1. Into a beaker weigh out the pigments in Part C and begin mixing.

2. Into a separate beaker, weigh out deionized water and butylene glycol in Part A and begin mixing. Sift in the Aristoflex slowly. Once all the polymer has been added, add the Disodium EDTA and begin heating to 75° C.

3. Weigh out Part B1 and heat to 80° C. Weigh out Part B2 and heat to 70° C. until clear. Add Part B2 to Part B1 to make Part B.

4. Add Part B to Part A and mix for 10 minutes at 75° C.-80° C.

5. After ten minutes, begin cooling the mixture of Part A and Part B to 55° C. At 55° C. or below add the pigment mixture (Part C), followed by the water rinse.

6. Weigh out Part D into a separate beaker and mix well. Once the mixture of Parts A, B and C has cooled to 45° C., add Part D.

7. Weigh out the mica slurry (Part E) into a separate beaker. Add Part E, to the mixture of Parts A, B, C and D at 40-45° C.

8. Add the fragrance and DMDM in Part F individually to the mixture of Parts A, B, C, D and E at 40-45° C.

Exemplary Composition for Medium Skin Tones

An exemplary composition for medium skin tones is set forth in the following table.

| PART | DESCRIPTION | % wt. |
|---|---|---|
| A | Deionized Water | 64.965% |
| | Butylene Glycol | 3.000% |
| | Disodium EDTA | 0.050% |
| | Aristoflex AVC (ammonium | 0.350% |
| | acryloyldimethyltaurate/VP copolymer) | |
| | Propylparaben NF | 0.100% |
| | Methylparaben | 0.200% |
| B | Glyceryl Stearate & PEG 100 Stearate | 3.500% |
| | Cetearyl Alcohol | 3.500% |
| | Ceteareth-20 | 0.500% |
| | Cetyl Alcohol | 0.500% |
| | Abil EM-90 (Cetyl PEG/PPG-10/1 Dimethicone) | 0.400% |
| | Bernel Ester DID (Diisopropyl Dimer Dilinoleate) | 0.500% |
| | Finsolv TN (C12-15 Alkyl Benzoate) | 3.000% |
| C | Deionized Water | 4.000% |
| | Iron Oxide (Kobo Yellow WSJ20EYAMP) | 0.040% |
| | Iron Oxide (Kobo Red WSJ22ERAMP) | 0.018% |
| | Iron Oxide (Kobo Black) | 0.007% |
| | Deionized Water (Rinse) | 1.000% |
| D | Koboguard 5400 IDD (Hydrogenated | 2.500% |
| | Polycyclopentadiene and Isododecane) | |
| | Permethyl 101A (isohexadecane) | 4.500% |
| | Dimethicone 200 5 CS | 0.750% |
| | Gransil KO (dimethicone and cyclopentasiloxane | 0.300% |
| | and polysilicone-11) | |
| E | Deionized Water | 4.000% |
| | Timica Silkwhite 110W (Mica and | 0.120% |
| | Titanium Dioxide) | |
| | Gemtone Amber G001 (Mica and Titanium Dioxide | 0.100% |
| | and Iron Oxides) | |
| | Gemtone Tan Opal (Mica and Titanium Dioxide and | 0.100% |
| | Iron Oxides) | |
| | Desert Ref. Sunlit Cactus 862Z | 0.100% |
| | Deionized Water | 1.000% |
| F | DMDM Hydantoin | 0.400% |
| | Fragrance | 0.500% |
| | | 100.000% |

The above composition can be made by a procedure as set forth below.

1. Into a beaker weigh out the pigments in Part C and begin mixing.

2. Into a separate beaker, weigh out deionized water and Butylene glycol in Part A and begin mixing. Sift in the Aristoflex slowly. Once all the polymer has been added, add the Disodium EDTA and begin heating to 75° C.

3. Weigh out Part B and heat to 80° C.

4. Add Part B to Part A and mix for 10 minutes at 75° C.-80° C.

5. After ten minutes, begin cooling the mixture of Parts A and B to 55° C. At 55° C. or below, add the pigment mixture (Part C), followed by the water rinse.

6. Weigh out Part D into a separate beaker and mix well. Once the mixture of Parts A, B and C has cooled to 45° C., add Part D.

7. Weigh out the mica slurry (Part E) into a separate beaker. Add Part E, to the mixture of Parts A, B, C and D at 40-45° C.

8. Add the fragrance and DMDM in Part F individually to the mixture of Parts A, B, C, D and E at 40-45° C.

Exemplary Composition for Tan Skin Tones

An exemplary composition for tan skin tones is set forth in the following table.

| PART | DESCRIPTION | % wt. |
|---|---|---|
| A | Deionized Water | 65.178% |
| | Butylene Glycol | 3.000% |
| | Disodium EDTA | 0.050% |
| | Aristoflex AVC (ammonium | 0.350% |
| | acryloyldimethyltaurate/VP copolymer) | |
| | Propylparaben NF | 0.100% |
| | Methylparaben | 0.200% |
| B | Glyceryl Stearate & PEG 100 Stearate | 3.500% |
| | Cetearyl Alcohol | 3.500% |
| | Ceteareth-20 | 0.500% |
| | Cetyl Alcohol | 0.500% |
| | Abil EM-90 (Cetyl PEG/PPG-10/1 Dimethicone) | 0.400% |
| | Bernel Ester DID (Diisopropyl Dimer Dilinoleate) | 0.500% |
| | Octyldodecyl Myristate | 1.500% |
| | Ethylhexyl Isonoate | 1.500% |
| C | Deionized Water | 4.000% |
| | Iron Oxide (Kobo Yellow WSJ20EYAMP) | 0.030% |
| | Iron Oxide (Kobo Red WSJ22ERAMP) | 0.015% |
| | Iron Oxide (Kobo Black) | 0.007% |
| | Deionized Water (Rinse) | 1.000% |
| D | Koboguard 5400 IDD (Hydrogenated | 2.500% |
| | Polycyclopentadiene and Isododecane) | |
| | Permethyl 101A (isohexadecane) | 4.500% |
| | Dimethicone 200 5CS | 0.750% |
| | Gransil KO (dimethicone and cyclopentasiloxane | 0.300% |
| | and polysilicone-11) | |
| E | Deionized Water | 4.000% |
| | Gemtone Amber G001 (Mica and Titanium Dioxide | 0.350% |
| | and Iron Oxides) | |
| | KTZ Interfine Gold [Mica and Titanium Dioxide | 0.070% |
| | (CI 77891) and Tin oxide and Triethoxy | |
| | Caprylylsilane] | |
| | Deionized Water | 1.000% |
| F | DMDM Hydantoin | 0.400% |
| | Fragrance | 0.300% |
| | | 100.000% |

The above composition can be made by a procedure as set forth below.

1. Into a beaker weigh out the pigments in Part C and begin mixing.

2. Into a separate beaker, weigh out deionized water and Butylene glycol in Part A and begin mixing. Sift in the Aristoflex slowly. Once all the polymer has been added, add the Disodium EDTA and begin heating to 75° C.

3. Weigh out Part B and heat to 80° C.

4. Add Part B to Part A and mix for 10 minutes at 75° C.-80° C.

5. After ten minutes, begin cooling the mixture of Parts A and B to 55° C. At 55° C. or below add the pigment mixture (Part C), followed by the water rinse.

6. Weigh out Part D into a separate beaker and mix well. Once the mixture of Parts A, B and C has cooled to 45° C., add Part D.

7. Weigh out the mica slurry (Part E) into a separate beaker. Add Part E to the mixture of Parts A, B, C and D at 40-45° C.

8. Add the fragrance and DMDM in Part F individually to the mixture of Parts A, B, C, D and E at 40-45° C.

Exemplary Composition for Deep Skin Tones

An exemplary composition for deep skin tones is set forth in the following table.

| PART | DESCRIPTION | % wt. |
|---|---|---|
| A | Deionized Water | 60.185% |
| | Butylene Glycol | 3.000% |
| | Disodium EDTA | 0.050% |
| | Aristoflex AVC (ammonium | 0.350% |
| | acryloyldimethyltaurate/VP copolymer) | |
| | Propylparaben NF | 0.100% |
| | Methylparaben | 0.200% |
| B | Glyceryl Stearate & PEG 100 Stearate | 3.500% |
| | Cetearyl Alcohol | 3.500% |
| | Ceteareth-20 | 0.500% |
| | Cetyl Alcohol | 0.500% |
| | Abil EM-90 (Cetyl PEG/PPG-10/1 Dimethicone) | 0.400% |
| | Bernel Ester DID (Diisopropyl Dimer Dilinoleate) | 0.500% |
| | Butyrospermum parkii (Shea butter) | 5.000% |
| | Octyldodecyl Myristate | 1.500% |
| | Ethylhexyl Isonoate | 1.500% |
| C | Deionized Water | 4.000% |
| | Iron Oxide (Kobo Yellow WSJ20EYAMP) | 0.120% |
| | Iron Oxide (Kobo Red WSJ22ERAMP) | 0.068% |
| | Iron Oxide (Kobo Black) | 0.027% |
| | Deionized Water | 1.000% |
| D | Koboguard 5400 IDD (Hydrogenated | 2.500% |
| | Polycyclopentadiene and Isododecane) | |
| | Permethyl 101A (isohexadecane) | 4.500% |
| | Dimethicone 200 5CS | 0.750% |
| | Gransil KO (dimethicone and cyclopentasiloxane | 0.300% |
| | and polysilicone-11) | |
| E | Deionized Water | 4.000% |
| | Cloisonne Monarch Gold 233X | 0.100% |
| | Cloisonne Nu Antique Copper 340XB | 0.050% |
| | Cloisonne Satin Bronze | 0.050% |
| | Deionized Water | 1.000% |
| F | DMDM Hydantoin | 0.400% |
| | Fragrance | 0.35% |
| | | 100.00% |

The above composition can be made by a procedure as set forth below.

1. Into a beaker weigh out the pigments in Part C and begin mixing.

2. Into a separate beaker, weigh out deionized water and Butylene glycol in Part A and begin mixing. Sift in the Aristoflex slowly. Once all the polymer has been added, add the Disodium EDTA and begin heating to 75° C.

3. Weigh out Part B and heat to 80° C.

4. Add Part B to Part A and mix for 10 minutes at 75° C.-80° C.

5. After ten minutes, begin cooling the mixture of Parts A and B to 55° C. At 55° C. or below add the pigment mixture (Part C), followed by the water rinse.

6. Weigh out Part D into a separate beaker and mix well. Once the mixture of Parts A, B and C has cooled to 45° C., add Part D.

7. Weigh out the mica slurry (Part E) into a separate beaker. Add Part E, to the mixture of Parts A, B, C and D at 40-45° C.

8. Add the fragrance and DMDM in Part F individually to the mixture of Parts A, B, C, D and E at 40-45° C.

Second Additional Exemplary Composition for Fair Skin Tones

A second additional exemplary composition for fair skin tones is set forth below.

| | | | Target |
|---|---|---|---|
| PART | DESCRIPTION | % wt. | Wt. (g) |
| A | Deionized Water | 63.040% | 630.4000 |
| | Butylene Glycol | 3.000% | 30.0000 |
| | Disodium EDTA | 0.050% | 0.5000 |
| | Propylparaben NF | 0.100% | 1.0000 |
| | Methylparaben | 0.200% | 2.0000 |
| B | Glyceryl Stearate & PEG 100 Stearate | 3.500% | 35.0000 |
| | Cetearyl Alcohol | 3.500% | 35.0000 |
| | Ceteareth-20 | 0.500% | 5.0000 |
| | Cetyl Alcohol | 0.500% | 5.0000 |
| | Cetyl PEG/PPG10-1 Dimethicone | 0.400% | 4.0000 |
| | Diisopropyl Dimer Dilinoleate | 0.500% | 5.0000 |
| | Isopropyl Palmitate | 1.000% | 10.0000 |
| | C12-15 Alkyl Benzoate/Finsolv TN | 2.000% | 20.0000 |
| | Ammonium Acryloyldimethyltaurate/ | 0.350% | 3.5000 |
| | VP Copolymer | | |
| | Titanium Dioxide (and) Stearic Acid | 1.500% | 15.0000 |
| | (and) Alumina | | |
| | BHT FCC | 0.020% | 0.2000 |
| C | Hydrogenated Polycylcopentadiene | 2.500% | 25.0000 |
| | Isohexadecane | 4.500% | 45.0000 |
| | Dimethicone 200 (5CST) | 1.000% | 10.0000 |
| | Gransil KO | 0.300% | 3.0000 |
| D | Deionized Water | 4.000% | 40.0000 |
| | Diazolidinyl Urea | 0.100% | 1.0000 |
| | Kobo Yellow WSJ20EYAMP | 0.050% | 0.5000 |
| | Kobo Red WSJ22ERAMP | 0.020% | 0.2000 |
| | Kobo Black | 0.010% | 0.1000 |
| | Deionized Water (Rinse) | 1.000% | 10.0000 |
| E | Deionized Water | 4.000% | 40.0000 |
| | Titanium Dioxide (and) Mica | 0.340% | 3.4000 |
| | Titanium Dioxide (and) Mica | 0.170% | 1.7000 |
| | Deionized Water | 1.000% | 10.0000 |
| F | DMDM Hydantoin | 0.400% | 4.0000 |
| G | Fragrance | 0.450% | 4.5000 |
| | | 100.00% | 1000.000 |

The above composition can be made by a procedure as set forth below.

1. In a container, weigh out the pigments in Part D and begin mixing (e.g., using a lightning mixer).

2. Into a separate container, weigh out deionized water and butylene glycol in Part A and begin heating to 75° C. Add Disodium EDTA and parabens at ˜50° C.-60° C.

3. Weigh out Part B into a separate container and heat to 80° C. After the oil phase has melted down, add Ammonium Acryloyldimethyltaurate/VP Copolymer and disperse with moderate mixing (e.g., using a lightning mixer). After the polymer has been dispersed, add the TiO₂ powder and mix for 5-10 minutes until completely dispersed.

4. Once Part A has reached 75° C., add Part B and mix for 10 minutes at 75° C.-80° C. Increase agitation and mix for five minutes to ensure good emulsification.

5. After five minutes, begin cooling the mixture of Parts A and B to 55° C.

6. Weigh out Part C into a separate beaker and mix well (e.g., using a lightning mixer). Once the mixture of Parts A and B has cooled to 55° C., add Part C to the mixture of Parts A and B.

7. After ten minutes, add Part D to the mixture of Parts A, B and C at 55° C.-50° C.

8. Weigh out the mica slurry (Part E) into a separate beaker. Add Part E, to the mixture of Parts A, B, C and D at 50-45° C.

9. Add the fragrance and DMDM in Parts F and G individually to the mixture of Parts A, B, C, D and E at 40° C.

Third Additional Exemplary Compositions for Fair Skin Tones

A third additional exemplary composition for fair skin tones is set forth below.

| | | | Target |
|---|---|---|---|
| PART | DESCRIPTION | % wt. | Wt. (g) |
| A | Deionized Water | 59.641% | 596.4000 |
| | Butylene Glycol | 3.000% | 30.0000 |
| | Disodium EDTA | 0.050% | 0.5000 |
| | Propylparaben NF | 0.100% | 1.0000 |
| | Methylparaben NF | 0.200% | 2.0000 |
| B | Glyceryl Stearate & PEG 100 Stearate | 2.000% | 20.0000 |
| | Cetearyl Alcohol | 2.500% | 25.0000 |
| | Ceteareth-20 | 1.500% | 15.0000 |
| | Cetyl Alcohol NF | 0.500% | 5.0000 |
| | Cetyl PEG/PPG10-1 Dimethicone | 0.400% | 4.0000 |
| | Diisopropyl Dimer Dilinoleate | 0.500% | 5.0000 |
| | Dicaprylyl Ether | 2.000% | 20.0000 |
| | Petrolatum White USP | 3.500% | 35.0000 |
| | Silica | 1.667% | 16.6700 |
| | BHT FCC | 0.020% | 0.2000 |
| | Ammonium Acryloyldimethyltaurate/ | 0.500% | 5.0000 |
| | VP Copolymer (Aristoflex AVC) | | |
| C | Hydrogenated Polycylcopentadiene | 2.000% | 20.0000 |
| | Isohexadecane | 3.600% | 36.0000 |
| | Dimethicone 200 CST | 1.000% | 10.0000 |
| | Gransil KO | 0.300% | 3.0000 |
| D | Deionized Water | 4.000% | 40.0000 |
| | Diazolidinyl Urea | 0.100% | 1.0000 |
| | WSJ28PFAMP | 3.333% | 33.3300 |
| | Kobo Yellow WSJ20EYAMP | 0.150% | 1.5000 |
| | Kobo Red WSJ22ERAMP | 0.060% | 0.6000 |
| | Kobo Black | 0.030% | 0.3000 |
| | Deionized Water (Rinse) | 1.000% | 10.0000 |
| E | Deionized Water | 4.000% | 40.0000 |
| | Titanium Dioxide (and) Mica | 0.330% | 3.3000 |
| | Titanium Dioxide (and) Mica | 0.170% | 1.7000 |
| | Deionized Water | 1.000% | 10.0000 |
| F | DMDM Hydantoin | 0.400% | 4.0000 |
| G | Fragrance | 0.450% | 4.5000 |
| | | 100.00% | 1000.000 |

The above composition can be made by a procedure as set forth below.

1. Into a beaker weigh out the pigments in Part D and begin mixing (e.g., using a lightning mixer).

2. Into a separate beaker, weigh out deionized water and Butylene glycol in Part A and begin heating to 75° C. Add Disodium EDTA and parabens at ˜50° C.-60° C.

3. Weigh out Part B into a separate beaker and heat to 80° C. After oil phase has melted down, add Ammonium Acryloyldimethyltaurate/VP Copolymer and disperse with moderate mixing (e.g., using a lightning mixer) for 5-10 min. After all the polymer has been dispersed, add the silica and disperse for 5-10 min.

4. Once Part A has reached 75° C., add Part B and mix for 10 minutes at 75° C.-80° C. Increase agitation and mix for five minutes to ensure good emulsification.

5. After five minutes, begin cooling the mixture of Parts A and B to 55° C.

6. Weigh out Part C into a separate beaker and mix well using a lightning mixer. Once the mixture of Parts A and B has cooled to 55° C., add Part C to the mixture of Parts A and B.

7. After ten minutes, add Part D to the mixture of Parts A, B and C at 55° C.-50° C.

8. Weigh out the mica slurry (Part E) into a separate beaker. Add Part E, to the mixture of Parts A, B, C and D at 50° C.-45° C.

9. Add the fragrance and DMDM in Parts F and G individually to the mixture of Parts A, B, C, D and E at 40° C.

Second Additional Exemplary Composition for Medium Skin Tones

A second additional exemplary composition for medium skin tones is set forth below.

| | | | Target |
|---|---|---|---|
| PART | DESCRIPTION | % wt. | Wt. (g) |
| A | Deionized Water | 66.38600% | 663.86 |
| | Propylparaben NF | 0.10000% | 1.00 |
| | Methylparaben | 0.20000% | 2.00 |
| B | Glycerin USP | 6.00000% | 60.00 |
| | Xanthan Gum | 0.10000% | 1.00 |
| C | Cetearyl Alcohol | 4.00000% | 40.00 |
| | Ceteareth-20 | 1.25000% | 12.50 |
| | Petrolatum White | 2.00000% | 20.00 |
| | Mineral Oil Light NF | 0.75000% | 7.50 |
| | C12-15 Alkyl Benzoate | 0.75000% | 7.50 |
| | Octyldodecyl Myristate | 0.30000% | 3.00 |
| | Ethylhexyl Isononanoate | 0.50000% | 5.00 |
| | Cetyl Esters Wax NF | 0.00000% | — |
| | Glyceryl Stearate & PEG-100 | 0.50000% | 5.00 |
| | Stearate | | |
| | Hydroxyethyl Acrylate/Sodium | 0.65000% | 6.50 |
| | Acryloyldimethyl Taurate Copolymer | | |
| | (Sepinov EMT) | | |
| | Polypropylene (Mattewax 511) | 1.66000% | 16.60 |
| D | Dimethicone (10 CST) | 1.25000% | 12.50 |
| E | Deionized Water | 4.00000% | 40.00 |
| | WSJ28PFAMP | 2.50000% | 25.00 |
| | WSJ22EYAMP - Kobo Yellow | 0.16000% | 1.60 |
| | WSJ22ERAMP - Kobo Red | 0.03000% | 0.30 |
| | WSJ20EBAMP - Kobo Black | 0.01400% | 0.14 |
| | Deionized Water | 1.00000% | 10.00 |
| F | Deionized Water | 3.00000% | 30.00 |
| | Mica (and) Titanium Dioxide (and) | 0.75000% | 7.50 |
| | Iron Oxides | | |
| | Mica (and) Iron Oxides (and) | 0.25000% | 2.50 |
| | Titanium Dioxides | | |
| | Deionized Water | 1.00000% | 10.00 |
| G | DMDM Hydantoin | 0.40000% | 4.00 |
| H | Fragrance | 0.50000% | 5.00 |
| | | 100.00000% | 1,000.00 |

The above composition can be made by a procedure as set forth below.

1. Weigh out Part A into a container and begin heating to 75° C.-80° C. Begin dipersing the Xanthun Gum (Part B) in the Glycerin and add to Part A. Heat the mixture of Parts A and B to 75C.-80° C.

2. Weigh out Part C and begin heating to 80° C. Once the oil phase has melted, add Sepinov EMT and begin mixing with moderate agitation for 5-10 min. until thoroughly dispersed. Next, add in the Mattewax 511 and mix for 5-10 min. until dispersed.

3. Add Part C to the mixture of Parts A and B once the mixture of Parts A and B has reached 75° C.-80° C. Increase agitation and mix for five minutes to ensure good emulsification.

4. After five minutes, begin cooling the mixture of Parts A, B and C to 60° C.

5. Weigh out Part D into a separate container and add to the mixture of Parts A, B and Cat 60° C.

6. Weigh out Part E into a separate container and begin mixing (e.g., using a lightning mixer). At about 55° C., add Part E to the mixture of Parts A, B, C and D.

7. Weigh out and add Parts F, G, and H individually to the mixture of Parts A, B, C and D.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A cosmetic composition for application to the skin comprising:
one or more pigments coated with a composition comprising an acrylic polymer;
an emulsifier;
optionally, one or more emollients; and
optionally, one or more organic sunscreen agents;
wherein each of the one or more pigments is present in the composition at a concentration such that the composition, when applied to the skin of a user, imparts a pigment to the skin which masks skin imperfections and imparts a more uniform tone to the skin.

2. The composition of claim 1, wherein the acrylic polymer comprises an ethyl methacrylate/n-butyl acrylate/2-methylhexyl acrylate copolymer.

3. The composition of claim 1, wherein the composition imparts a tone to the skin which corresponds to the unexposed skin color of one of the Fitzpatrick skin phototypes I-VI.

4. The composition of claim 1, wherein the composition further comprises a film forming agent.

5. The composition of claim 1, wherein the composition comprises one or more organic sunscreen agents.

6. The composition of claim 1, further comprising a reflective pigment.

7. The composition of claim 1, further comprising a siloxane compound having the following general structure: 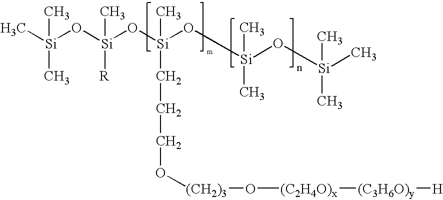
wherein R is an alkyl group and wherein m, n, x and y are integers.

8. The composition of claim 1, wherein the emulsifier is selected from the group consisting of glyceryl stearate, PEG-100 stearate, cetearyl alcohol, ceteareth-20 and combinations thereof.

9. A method comprising applying a cosmetic composition as set forth in claim 1 to the skin.

10. A cosmetic composition for application to the skin comprising:
one or more pigments;
an emulsifier;
optionally, one or more emollients;
a film forming agent; and
optionally, one or more organic sunscreen agents;
wherein each of the one or more pigments are present in the composition at a concentration such that the composition, when applied to the skin of a user, imparts a pigment to the skin which masks skin imperfections and imparts a more uniform tone to the skin.

11. The composition of claim 10, wherein the film forming agent comprises hydrogenated polycyclopentadiene.

12. The composition of claim 11, wherein the composition further comprises isohexadecane.

13. The composition of claim 10, wherein the composition comprises one or more organic sunscreen agents.

14. The composition of claim 10, wherein the composition imparts a tone to the skin which corresponds to the unexposed skin color of one of the Fitzpatrick skin phototypes I-VI.

15. The composition of claim 10, wherein the pigments are coated with a composition comprising an acrylic polymer.

16. The composition of claim 10, further comprising a reflective pigment.

17. The composition of claim 10, further comprising a compound having the following general structure: 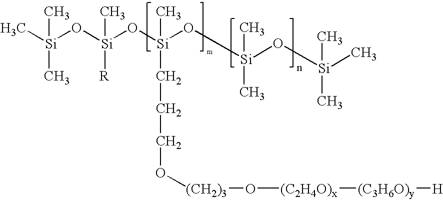
wherein R is an alkyl group and wherein m, n, x and y are integers.

18. The composition of claim 10, wherein the emulsifier is selected from the group consisting of glyceryl stearate, PEG-100 stearate, cetearyl alcohol, ceteareth-20 and combinations thereof.

19. A method comprising applying a cosmetic composition as set forth in claim 10 to the skin.

20. A cosmetic composition for application to the skin comprising:
one or more pigments other than mica in a concentration of less than 0.09 percent by weight based on the total weight of the composition;
an emulsifier;
optionally, one or more emollients; and
optionally, one or more organic sunscreen agents;
wherein each of the one or more pigments are present in the composition at a concentration such that the composition, when applied to the skin of a user, imparts a pigment to the skin which masks skin imperfections and imparts a more uniform tone to the skin.

21. The composition of claim 20, wherein the one or more pigments are selected from the group consisting of hydrated ferric oxide (FeHO₂), ferric oxide (Fe₂O₃), ferrosoferric oxide (Fe₃O₄) and combinations thereof.

22. The composition of claim 20, wherein the one or more pigments are coated with a composition comprising an acrylic polymer

23. The composition of claim 20, wherein the composition imparts a tone to the skin which corresponds to the unexposed skin color of one of the Fitzpatrick skin phototypes I-VI.

24. The composition of claim 20, wherein the composition further comprises a film forming agent.

25. The composition of claim 24, wherein the film forming agent comprises hydrogenated polycyclopentadiene.

26. The composition of claim 25, wherein the composition further comprises isohexadecane.

27. The composition of claim 20, wherein the composition further comprises one or more organic sunscreen agents.

28. The composition of claim 20, further comprising a reflective pigment.

29. The composition of claim 20, further comprising a compound having the following general structure: 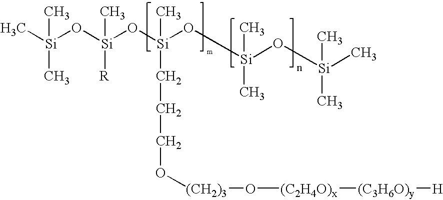
wherein R is an alkyl group and wherein m, n, x and y are integers.

30. The composition of claim 20, wherein the emulsifier is selected from the group consisting of glyceryl stearate, PEG-100 stearate, cetearyl alcohol, ceteareth-20 and combinations thereof.

31. A method comprising applying a cosmetic composition as set forth in claim 20 to the skin.

32. A method of making an oil-in-water emulsion comprising:
mixing an aqueous phase with an oil phase comprising an oil-in-water emulsifier and, optionally, one or more emollients at a temperature greater than 70° C.;
cooling the resulting mixture;
mixing one or more iron oxide pigments into the resulting mixture at a temperature at or below 60° C.; and
mixing hydrogenated polycyclopentadiene and isohexadecane into the resulting mixture at a temperature at or below 55° C.

33. The method of claim 32, further comprising mixing one or more reflective pigments into the mixture subsequent to mixing hydrogenated polycyclopentadiene and isohexadecane into the resulting mixture.

34. The method of claim 32, wherein the aqueous phase, prior to mixing with the oil phase, comprises butylene glycol and an ammonium acryloyldimethyltaurate/vinyl pyrrolidone copolymer.

35. The method of claim 32, wherein the iron oxide pigments include hydrated ferric oxide (FeHO₂), ferric oxide (Fe₂O₃), ferrosoferric oxide (Fe₃O₄).

36. The method of claim 32, wherein the iron oxide pigments are coated with a composition comprising an acrylic polymer.

37. The method of claim 36, wherein the acrylic polymer comprises an ethyl methacrylate/n-butyl acrylate/2-methylhexyl acrylate copolymer.

38. The method of claim 33, wherein the reflective pigments comprise mica particles.

39. The method of claim 38, wherein the mica particles comprise mica particles coated with iron oxide and/or mica particles coated with titanium dioxide.

40. The method of claim 32, wherein the oil-in-water emulsifier is selected from the group consisting of glyceryl stearate, PEG-100 stearate, cetearyl alcohol, ceteareth-20 and combinations thereof.

41. The method of claim 32, wherein the oil phase further comprises, prior to mixing with the aqueous phase, a compound having the following general structure: 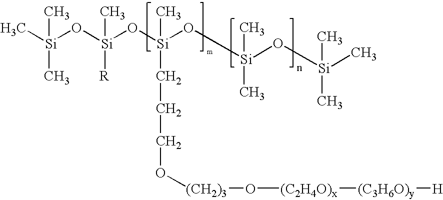
wherein R is an alkyl group and wherein m, n, x and y are integers.

42. The method of claim 41, wherein R is a cetyl group.

43. The method of claim 41, wherein the emulsion comprises less than 1 weight % of the siloxane compound or less than 0.5 weight % of the siloxane compound based on the total weight of the composition.

44. The method of claim 32, wherein the oil phase further comprises, prior to mixing with the aqueous phase, one or more emollients selected from the group consisting of diisopropyl dimer dilinoleate, C₁₂₋₁₅ alkyl benzoates, octyldodecyl myristate, ethylhexyl myristate, shea butter and combinations thereof.

45. The method of claim 32, wherein the iron oxide pigments are mixed into the resulting mixture at a temperature of 53° C.-57° C.

46. The method of claim 32, wherein the hydrogenated polycyclopentadiene and isohexadecane are mixed into the resulting mixture at a temperature of 50° C.-55° C.
